(19) United States
(12) Patent Application Publication
(10) Pub. No.: US 2005/0113298 A1
(43) Pub. Date: May 26, 2005

(54) Receptor binding peptides derived from the SARS S protein

(75) Inventors: Michael R. Farzan, Cambridge, MA (US); Wenhui Li, Boston, MA (US)

Correspondence Address: FITCH, EVEN, TABIN & FLANNERY, P. O. BOX 65973, WASHINGTON, DC, 20035, US

(73) Assignee: The Brigham and Women's Hospital, Inc.

(21) Appl. No.: 10/939,113

(22) Filed: Sep. 13, 2004

Related U.S. Application Data
(60) Provisional application No. 60/502,610, filed on Sep. 15, 2003.

(51) Int. Cl. A61K 38/17; C07H 21/04; C07K 14/705; C07K 16/40

(52) U.S. Cl. 514/12; 530/350; 530/388.26; 435/69.1; 435/320.1; 435/325; 536/23.5

(57) ABSTRACT

The present invention is directed to peptides that bind to the cellular receptors for the SARS S protein. The invention also includes polynucleotides coding for these peptides and methods in which they can be used to block the binding of the S protein to receptor. The peptides can also be detectably labeled and used in assays for identifying cells that have receptors for the S protein, in vaccines and for identifying other agents that inhibit receptor binding.

### CROSS REFERENCE TO RELATED APPLICATIONS

The present application claims the benefit of U.S. provisional application No. 60/502,610, filed on Sep. 15, 2003, which is incorporated in its entirety herein by reference.

### FIELD OF THE INVENTION

The present invention is directed to peptides which represent the receptor-binding region of the SARS S protein. The peptides may be used, inter alia, to block interactions between the receptor and the S protein, to identify cells expressing receptor and to search for inhibitors of binding.

### BACKGROUND OF THE INVENTION

Severe acute respiratory syndrome (SARS) is a highly contagious viral disease caused by SARS-associated coronavirus (Drosten, et al., N. Engl. J Med. 348:1967-1976 (2003)). The disease appears to have originated in the Guangdong Province of China in early November of 2002 and to have quickly spread to North America, South America, Europe and Asia (Riley, et al., Science 300:1961-1966 (2003)). According to the World Health Organization (WHO), during the period from February to July of 2003, 8,437 people worldwide became sick with SARS and, of these, nearly 10% (813) died. SARS typically begins with a high fever and other generalized symptoms, such as headache, an overall feeling of discomfort, and body aches. As the disease progresses, patients usually develop pronounced respiratory symptoms and pneumonia (Avendano, et al., Can. Med. Assoc. J. 168:1649-1660 (2003)). Although the SARS epidemic has been contained for the present, it may reemerge at any time in the future. Thus, improved diagnostic procedures, strategies for blocking the entry of virus into cells and the development of new therapies are of great importance.

The genome of the SARS virus has been completely sequenced (Marra, et al., Science 300:1394-1404 (2003), see, also GenBank Accession No. AY278741). Based on this information and on a knowledge of the life cycle of other coronaviruses, it is believed that entry into host cells by the SARS virus is mediated by a surface protein, designated as “S,” which is analogous to the HIV-1 envelope glycoprotein. Some coronaviruses cleave the S protein into two fragments called S1 (which mediates receptor binding) and S2 (which mediates fusion of the virus to the host cell membrane). In other coronaviruses, including the one causing SARS, the S protein is not cleaved. Nevertheless, the S1 and S2 domains can be identified and initial receptor binding is apparently still mediated by S1.

The virus causing SARS (SARS-CoV) does not belong to any of the previously defined genetic and serological coronavirus groups and the SARS-CoV S protein that mediates virus entry into receptor-bearing cells is also distinct from those of other coronaviruses (Marra, et al., Science 300:1399-1404 (2003); Rota, et al., Science 300:1394-1399 (2003); Kubo, et al., J. Virol; 68:5403-5410 (1994); Dveksler, et al., J. Virol. 65:6881-6891 (1991); Dveksler, et al., J. Virol. 67:1-8 (1993) Bonavia, et al., J. Virol. 77:2530-2538 (2003); Breslin, et al., J. Virol. 77:4435-4438 (2003); Yeager, et al., Nature 357:420-422 (1992)). Reflecting this difference, SARS-CoV does not utilize any previously identified coronavirus receptors to infect cells. Rather, angiotensin-converting enzyme 2 (ACE2) serves as a functional receptor for this virus (Li, et al., Nature 426:450-454) (2003)).

### SUMMARY OF THE INVENTION

The present invention is based on studies in which the portion of the S1 domain that binds to receptor has been localized to a particular region. These studies are described in the Examples section and have led to the conclusion that there are definite limitations on how far the SARS S protein (as shown in FIG. 1 and SEQ ID NO:1) can be truncated from its N terminus or C terminus and still maintain the ability to effectively bind to the ACE2 receptor. Specifically, a truncated peptide capable of effective binding must maintain, at a minimum, amino acids 326-491 and preferably amino acids 318-510. The first 11 amino acids of the S protein (again as shown in FIG. 1 and SEQ ID NO:1) is a signal sequence. Thus, beginning at the N terminus of the S protein, a peptide able to bind receptor must extend from amino acid 12 at least to amino acid 491 and preferably at least to amino acid 510. Beginning at the C terminus, the peptide must extend in the direction of the N terminus at least to amino acid 326 and preferably at least to amino acid 318.

In its first aspect, the invention is directed to a substantially pure ACE-2 binding peptide comprising an amino acid sequence matching a sequence shown in SEQ ID NO:1 and which begins, starting at the N terminus, at an amino acid greater than 317 and less than 327 (i.e. at an amino acids between 318 and 326 inclusive). The peptide sequence extends toward the C terminus as shown in SEQ ID NO:1 but goes no further than amino acid 672. In three preferred embodiments: the peptide begins at amino acid 318; extends to at least amino acid 510; and extends no further than amino acid 510. The most preferred peptide corresponds to amino acids 318-510 and is shown in FIG. 2 and as SEQ ID NO:2.

The invention also includes another group of substantially pure peptides that bind to ACE2 and which also have a sequence matching that of SEQ ID NO:1. The C terminus of these peptides terminates at an amino acid greater than 490 and less than 511 (i.e. at amino acids 491-510 inclusive) as shown in SEQ ID NO:1. The peptides extend toward the N terminus to at least amino acid 326 and no further than amino acid 12. In three preferred embodiments: the peptide terminates at amino acid 510; extends to at least amino acid 318; and extends no further than amino acid 318. All of the peptides of the invention may be fused to the Fc region of IgG1, particularly human IgG1. As discussed in the Examples section the fused peptides retain their ability to bind to receptor. The most preferred fusion peptide is Fc domain of human IgG1 fused to the peptide of SEQ ID NO:2. The most preferred orientation is with the peptide lying at the N terminal end of the fusion protein and with the Fc domain at the C-terminal end of the fusion protein, i.e., with the C terminal amino acid of the peptide joined to the N-terminal amino acid of the Fc domain.

The term “substantially pure” as used herein refers to a peptide or a polynucleotide that is essentially free from other contaminating components found associated with it in the virus such as other proteins, carbohydrates or lipids. One method for determining the purity of a protein or nucleic acid is by electrophoresing a preparation in a matrix such as polyacrylamide or agarose. Purity is evidenced by the appearance of a single band after staining.

The term “matching” as used above refers to peptides having the same contiguous amino acid sequence as that in SEQ ID NO:1. It will however be recognized by those of skill in the art that it is usually possible to modify the sequences of peptides without significantly changing their activity. In particular, it is usually possible to make a limited number of amino acid changes, e.g., affecting less than 10% of the total sequence, without significantly affecting activity. This is particularly true where conservative amino acid changes are made. Examples of conservative amino acid changes include substituting one neutral amino acid for another, exchanging one acidic amino acid for another and exchanging one basic amino acid for another. These types of alterations are well known in the art. To the extent that changes of this type are insubstantial, i.e., do not affect the ability of a peptide to bind with specificity to ACE2, they result in a peptide that is equivalent to a peptide having the exact sequence of SEQ ID NO:1. “Specificity of binding” is defined in this context as occurring in situations where a peptide has at least a hundredfold greater affinity for the SARS S receptor than for other, unrelated proteins not binding S as part of their biological function. Guidance concerning certain residues that are critical to peptide binding is provided in the Examples section below.

The receptor-binding peptides described above can be detectably labeled, i.e., they can be attached to some type of molecule that can be either directly or indirectly assayed using standard laboratory techniques. For example, peptides may be attached to a radioactive isotope such as ¹²⁵I or to a fluorescent tag such as fluoresceineisothiocyanate (FITC). Peptides may also be detectably labeled by expressing them as a fusion protein in which they are attached to a marker amino acid sequence. A “marker amino acid sequence” is one that binds with high affinity to an antibody or other compound permitting detection. For example, peptides may be fused to the Fc fragment of IgG and the fusion polypeptide subsequently detected using an FITC-labeled antibody that binds with high affinity to Fc.

In another aspect, the invention is directed to substantially pure polynucleotides that encode the peptides described above either alone or after they have been fused to a marker amino acid sequence. The invention also includes vectors in which there is a promoter operably linked to a sequence consisting of nucleotides coding for the peptide. The term “operably linked” means that the promoter and coding sequence are joined in a manner that allows them to carry out their normal functions, i.e., transcription of the coding sequence is under the control of the promoter and the transcript produced is correctly translated into the desired peptide. In addition, the invention includes host cells that have been transformed with these vectors.

In another aspect the invention is directed to methods for inhibiting the binding of the S protein of SARS to a host cell receptor by contacting the receptor with one or more of the peptides described above. The S protein and peptide must be concurrently available to the receptor to allow them to compete for binding. For example, in vitro, peptide and S protein might be mixed together in a solution and concurrently exposed to cells known to contain the SARS S receptor. It should also be appreciated that the S protein may also be present on the virus. Thus, peptide may be used to prevent interaction between virus and cells either in vitro or in vivo.

The methods discussed above can be adapted to either determine whether cells contain a SARS S receptor or to identify other compounds that bind to the receptor. In order to determine whether cells have a SARS S receptor, the cells under examination can be incubated in a solution containing detectably labeled peptide as described above. After incubation, the solution may be removed from the cells and they may then be assayed to determine the amount of peptide bound, i.e., by assaying for the label. Methods can include one or more wash steps to remove unbound peptide from cells prior to assay and nonspecific binding may corrected for using standard techniques. For example, nonspecific binding may be determined by performing incubations in which cells are combined with labeled peptide in the manner described above but in the presence of a large excess, e.g., a hundredfold excess, of unlabeled peptide. The amount of label detected in samples prepared in this manner may be subtracted from the incubations carried out in the absence of unlabeled peptide to determine “specific binding.”

In order to determine whether a test compound binds with specificity to the S protein receptor, incubations may be carried out with host cells that are known to express the receptor, e.g., Vero cells. Incubation should take place using solutions containing a detectably labeled peptide of the type described above and the test compound. After incubation, solution is removed and the cells may optionally be washed. They are then assayed to determine the amount of label present and the results obtained are compared to the results from cells prepared in the same manner but in which incubation is carried out in the absence of test compound. Ideally, the test compound should be examined at several different concentrations. Also, if desired, nonspecific binding may be corrected for in a manner such as that described above.

In preferred embodiments, the assays for determining the presence of receptor and for identifying agents that inhibit the binding of S protein to receptor are carried out using a fluorescence activated cell sorting (FACS) assay. The steps are the same as those described above except that peptides must be labeled in a manner that permits detection by fluorometry, e.g., with a compound such as FITC. Preferably, labeling is accomplished by expressing the peptide as a fusion protein in which it is joined to a marker amino acid sequence. In these cases, assays of bound peptide will involve a step in which a fluorescently tagged molecule is bound to the marker amino acid sequence on cells. Preferably, the marker amino acid sequence is the Fc fragment of IgG and is identified using a fluorescently labeled antibody, e.g., an antibody attached to FITC.

The assays described above for identifying whether a test compound inhibits the binding of the S protein to its receptor are also preferably performed using FACS methodology. Again, it is preferred that assays be performed using a solution in which peptide is fused to the Fc sequence of IgG, removing solution and then determining the amount of bound fusion protein by exposing cells to a fluorescently labeled antibody that recognizes and binds to the Fc sequence.

The peptides of the invention (or peptides fused to IGg) may be used to generate antibodies for use in some of the assays described above and to detect the presence of SARS. Techniques for this are well known in the art and involve injecting an immunocompetent animal such as a rabbit, horse goat, mouse etc. Antibodies may also be generated in people. In order to be of significant value, an antibody must bind to the SARS S protein with specificity, i.e., with at least a hundredfold greater affinity for SARS S than for other proteins.

### BRIEF DESCRIPTION OF THE DRAWINGS

FIG. 1: SARS S Amino Acid Sequence: FIG. 1 shows the full length amino acid sequence of the SARS S protein.

FIG. 2: Preferred Receptor Binding Peptide: FIG. 2 shows the most preferred peptide of the invention. It corresponds to amino acids 318-510 of SEQ ID NO:1.

FIG. 3: Residues 318 to 510 of the SARS-CoV S Protein Include the Receptor-Binding Domain: A: S1-Ig, containing S-protein residues 12-672 fused to the Fc region of human IgG1, or truncation variants of S1-Ig containing the indicated S-protein residues, were purified from media of transfected 293T cells. S1-Ig and variants were normalized for expression, as shown by Coomassie staining (top panel), and used to precipitate soluble metabolically labeled ACE2 (bottom panel). Precipitates were analyzed by SDS-PAGE, and ACE2 quantified by phosphorimaging. B: the indicated S1-Ig variants were incubated with ACE2-transfected 293T cells and analyzed by flow cytometry (bars with slanted lines), or used, as in (A), to immunoprecipitate soluble ACE2 (plain white bars). Bars indicate averages of two or more experiments normalized to results for S1-Ig. C: representation of truncation variants assayed in (A) and (B). Bars with slanted lines represent association with ACE2 greater than 25% of that observed for S1-Ig in both precipitation and flow-cytometry assays. Plain white bars represent ACE2 association less than 10%, in both assays, of that for S1-Ig. Arrow indicates 318-510 variant, the smallest fragment observed to bind ACE2.

FIG. 4: An S1-Ig Variant Containing Residues 318-510 Associates with ACE2 and Blocks S-Protein-Mediated Entry Better than does S1-Ig: A: S1-Ig, or variants containing residues 318-510 or 12-327, were purified from transfected 293T cells and quantified. An aliquot of each variant diluted to the indicated concentrations was visualized by SDS-PAGE and Coomassie staining (top panel), and used to precipitate soluble metabolically labeled ACE2. Precipitates were analyzed by SDS-PAGE and ACE2 was quantified by phosphorimaging. B: ACE2 precipitated by the indicated concentrations of S1-Ig and the indicated variants. An average of two experiments is shown. C: the indicated concentrations of S1-Ig, or of the 318-510 or 12-327 variants, were incubated with 293T cells expressing ACE2, together with an SIV modified to express green fluorescent protein (SIV-GFP) and pseudotyped with S protein of SARS-CoV or with VSV-G. Infection with pseudotyped virus was quantified by measuring green fluorescence by flow cytometry, and shown here as mean fluorescent intensity (m.f.i.). D: fluorescent microscopic fields of 293T cells transfected with ACE2, and incubated with SIV-GFP pseudotyped with S protein in the presence of 250 nM of the 12-327 (right) or the 318-510 (left) variants. Many microscopic fields of cells incubated with the 318-510 variant lacked observable fluorescing cells.

FIG. 5: Analysis of Point Mutations of S1-Ig and the 318-510 Variant. A: the 318-510 S1-Ig truncation variant, or variants thereof in which each of seven cysteines was altered individually to alanine, were analyzed as in FIG. 3. Variants in which cysteine 366 or 419 was altered to alanine did not express and were not further analyzed. A variant containing alterations of both cysteines 323 and 378 was also analyzed (right panel). B: 318-510 variants (left panel) or S1-Ig variants (right panel) in which glutamic acid 452 or aspartic acids 454, 463, or 480 were altered individually to alanine were analyzed as in FIG. 3. C: representation of the S proteins of SARS-CoV, HCoV-229E, and MHV, aligned by their S2 domains. Crosshatched bars represent leader and transmembrane sequences. Bars with slanted lines represent the receptor-binding domain. The receptor-binding domain of SARS-CoV is shown with N-glycosylation sites (small circles) and cysteines indicated. Residues that make a substantial contribution to ACE2 association (glutamic acid 452 and aspartic acid 454) are shown as plain white bars.

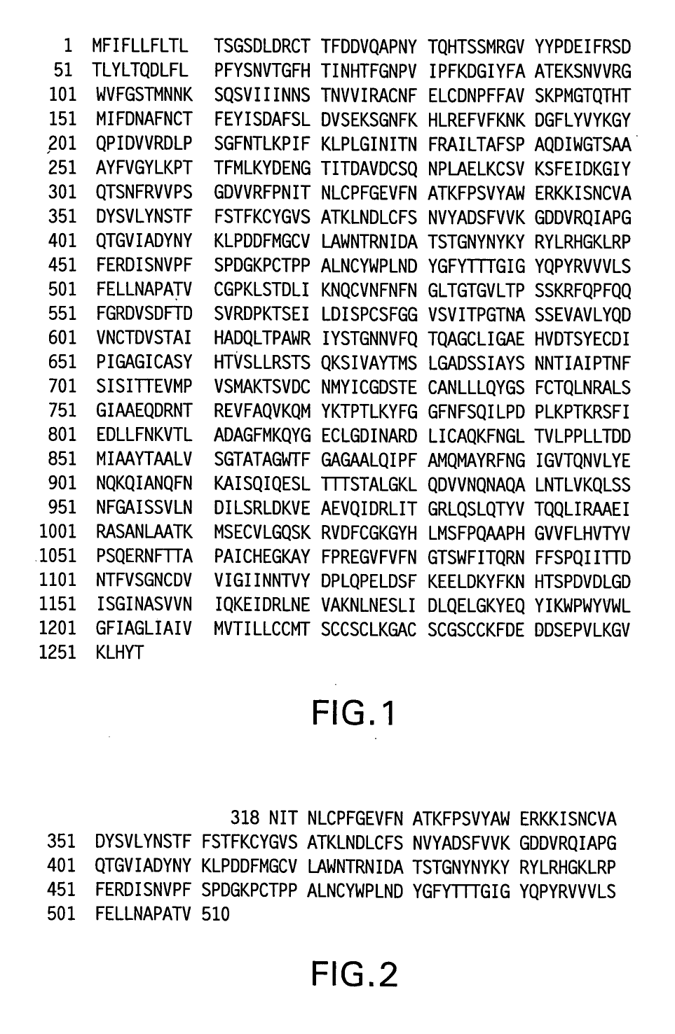

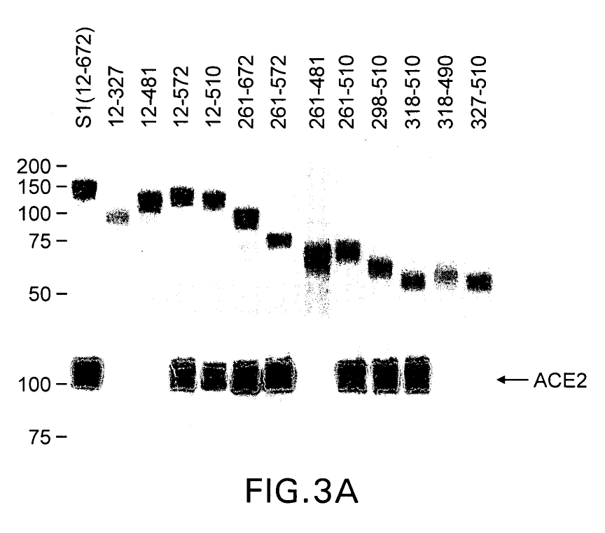

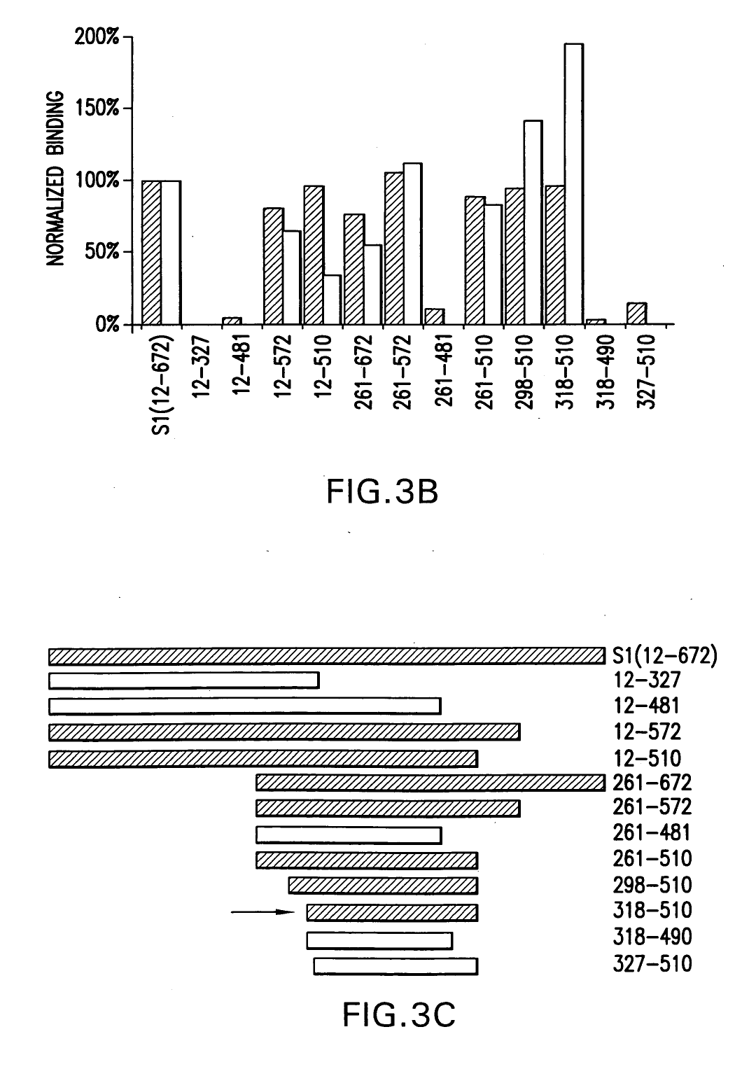

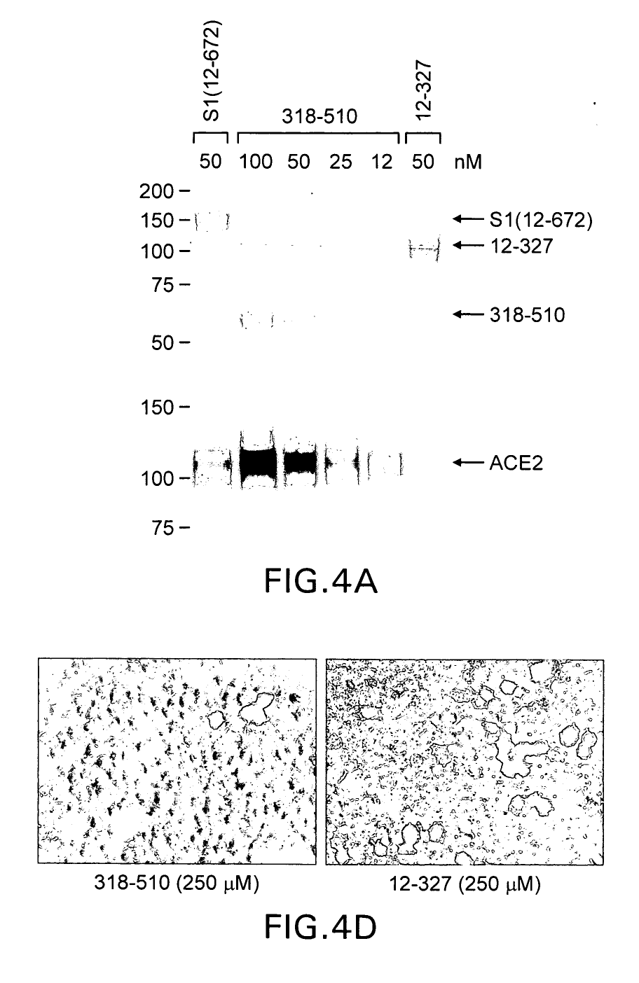

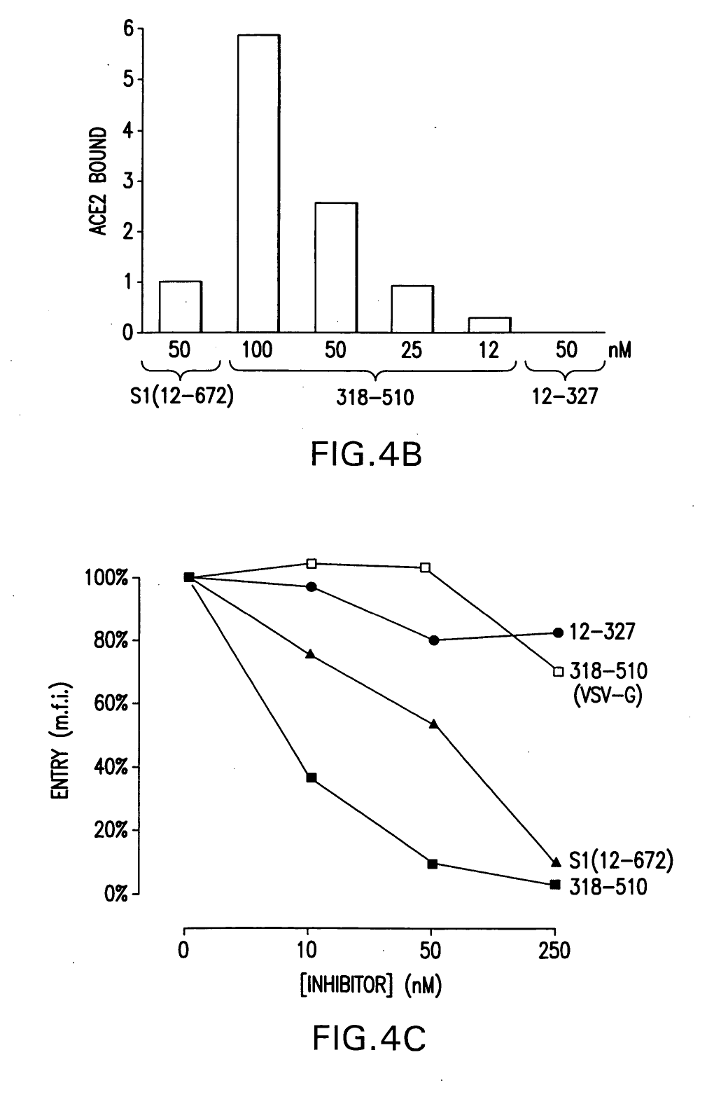

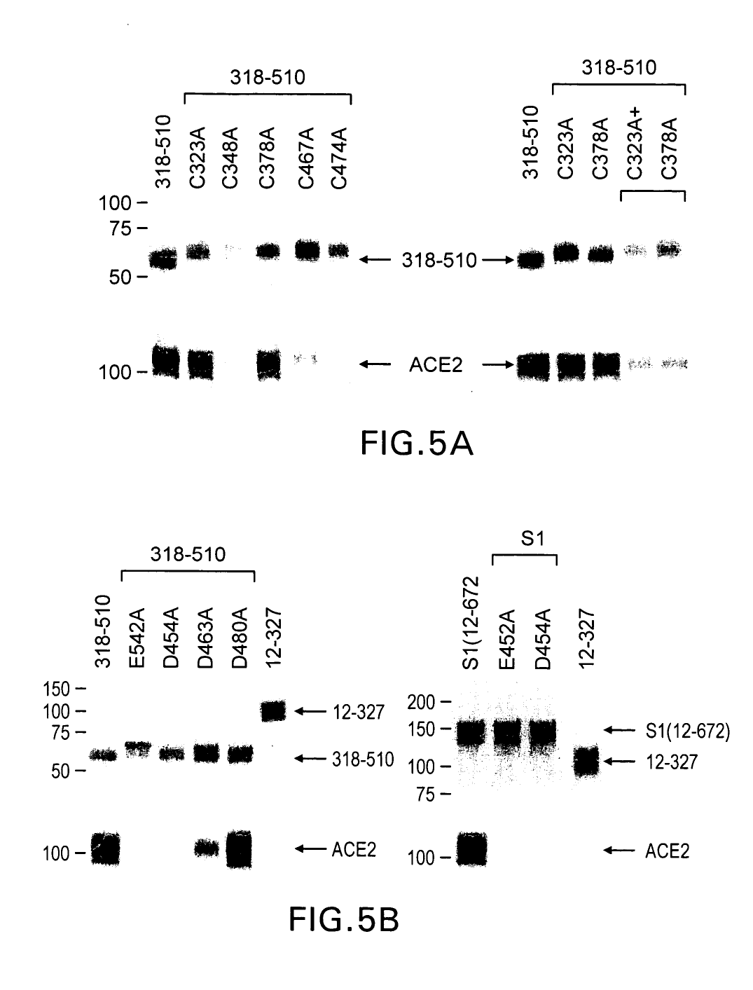

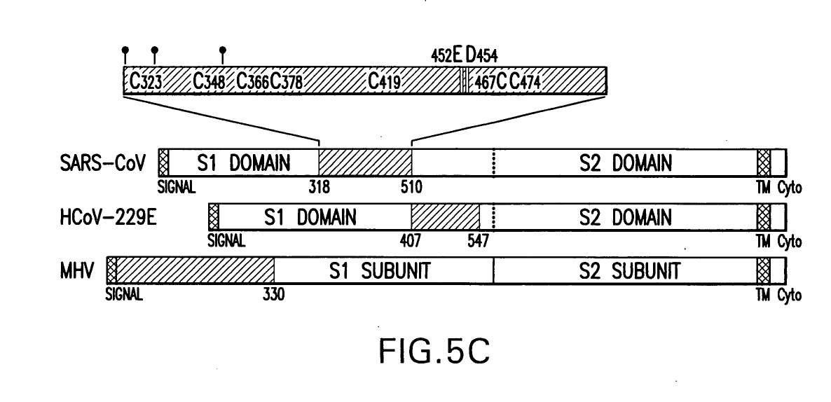

### DETAILED DESCRIPTION OF THE INVENTION

The present invention is based, in part, upon the identification of a specific region in the SARS S protein that binds to the S receptor. The discovery of peptides that compete for binding is important for several reasons. First, since interaction between the S protein and the host receptor is necessary for viral infection, the peptides may have therapeutic utility or, alternatively, they may be used in assays of the type described herein to identify other compounds of potential value in blocking infection. Experimentally, the peptides may be labeled and administered to test animals to identify cells that interact with SARS and in assays designed to identify cells that have receptors that bind to the S protein and which should therefore serve as a host for growing the virus. Finally, the peptides may be used to generate antibodies that cross-react with the S protein. These antibodies may be isolated and used diagnostically or they may be generated as a means of protecting against viral infection, i.e., the receptor-binding peptides may be used as part of a vaccine.

I. Peptides and Polynucleotides

The peptides of the invention may be made by any of method known in the art, with bacterial production or chemical synthesis being generally preferred. Purification can also be accomplished using standard procedures such as isolating peptides directly from resins used in solid state synthetic methods, using antibodies directed against the peptides, or by producing peptides in a form in which they are fused to a moiety that aids in purification and which can then be cleaved. In cases where peptides are used as part of a fusion protein, it may be convenient to produce them recombinantly. For example, nucleotides coding for the peptide may be ligated to a sequence coding for a marker protein, e.g., the Fc fragment of IgG. The fusion protein produced may then be isolated using the marker sequence. For example, in the case of a fusion protein involving Fc, purification may be accomplished using a column which has antibody that recognizes the Fc domain.

Any polynucleotide sequence coding for peptides may be used for their expression. Standard methods of molecular biology for producing vectors, transforming cells and recombinantly making protein may then be used.

II. Assay Methods

One of the main uses for peptides binding to the SARS S receptor is in assays designed to identify cells that have the receptor or in assays designed to identify agents that bind to the receptor and which may be used to block its interaction with the S protein. In the latter case, a source of the SARS S receptor is incubated together with detectably labeled peptide and with the compound being tested for binding activity. The preferred source for the receptor is Vero E6 cells, but other cells that express the receptor can also be used. After incubation of receptor, peptide and test compound, the receptor is separated from the solution containing the other components, e.g., by pelleting cells by centrifugation and then removing the supernatant. The amount of label remaining with the cells is then determined.

As noted above, the peptide used in assays must be detectably labeled in some manner, such as, with a radioisotope, a fluorescent label or chemiluminescent label. Examples of labels that may be used include ¹²⁵I, fluorescein, isothiocynate, rhodamine, fluorescamine, luminal and isoluminal. Most preferably, peptides are produced in the form of a fusion protein in which they are joined to a marker amino acid sequence such as that of the Fc IgG fragment. In this case, binding can be quantitated by performing a second incubation in which labeled antibody is allowed to bind to the marker sequence.

Nonspecific binding in assays may be determined by carrying out the binding reaction in the presence of a large excess of unlabeled peptide or fusion protein. For example, cells expressing the SARS S receptor may be incubated with labeled peptide and test compound in the presence of a hundredfold excess of unlabeled peptide. Nonspecific binding may be subtracted from total binding, i.e., binding in the absence of unlabeled peptide, to arrive at the specific binding for each sample tested. Other steps, such as washing, stirring, shaking, filtering and the like, may be included in the assays as necessary. Typically, wash steps are included after the separation of membrane-bound peptide from peptide remaining in solution and prior to quantitation. The specific binding obtained in the presence of the test compound is compared with that obtained in the presence of labeled peptide alone to determine the extent to which the test compound has displaced the peptide.

The assay described above can be varied in many different ways that are known in the art. For example, labeled peptide can be incubated with cells for the purpose of determining whether they express a SARS S receptor. In these assays, correction for nonspecific binding in the manner discussed above will be particularly important. All of these assays may also be performed using the intact S protein, the S1 domain or longer peptide sequences that are known to bind the receptor. It will also be desirable to test cells or compounds using the intact virus.

III. Antibodies to SARS S Receptor-Binding Peptides

Antibodies directed against the peptides described above may be produced in either people or animals. Isolated antibodies may be used diagnostically or experimentally, e.g., to isolate or quantitate either peptides or fusion proteins. Methods for making and detecting antibodies are well known to those of skill in the at as evidenced by standard reference works such as : Harlow, et al., Antibodies, A Laboratory Manual, Cold Spring Harbor Laboratory, N.Y. (1988); Kennett, et al., Monoclonal Antibodies and Hybridomas: A New Dimension in Biological Analyses (1980); Klein, Immunology: The Science of Self and Nonself Discrimination (1982); and Campbell, “Monoclonal Antibody Technology” in Laboratory Techniques in Biochemistry and Molecular Biology, (1984).

“Antibody” as used herein, is meant to include intact molecules as well as fragments which retain their ability to bind to antigen (e.g., Fab and F(ab)₂ fragments). These fragments are typically produced proteolytically by cleaving intact antibodies using enzymes such as papain (to produce Fab fragments) or pepsin (to produce F(ab)₂ fragments). The term “antibody” also refers to both monoclonal antibodies and polyclonal antibodies. Polyclonal antibodies are derived from the sera of animals immunized with the antigen. Monoclonal antibodies can be prepared using hybridoma technology (Kohler, et al., Nature 256:495 (1975); Hammerling, et al., in Monoclonal Antibodies in T Cell Hybridomas, Elsevier, N.Y. (1981)). In general, this technology involves immunizing an animal, usually a mouse, with antigen. The splenocytes of the immunized animals are extracted and fused with suitable myeloma cells, e.g., SP₂0 cells. After fusion, the resulting hybridoma cells are selectively maintained in HAT medium and then cloned by limiting dilution (Wands, et al., Gastroenterology 80:225-232 (1981)). The cells obtained through such selection are then assayed to identify clones which secrete antibodies capable of binding specifically to either peptide or S protein.

The antibodies or fragments of antibodies of the present invention may be used to detect the presence of S protein, either in a free state or attached to virus, using any of a variety of immunoassays. For example, the antibodies may be used in radioimmunoassay or immunometric assays, also known as “two site” or “sandwich” assays (see Chart, “An Introduction to Radioimmune Assay and Related Techniques,” in Laboratory Techniques in Biochemistry and Molecular Biology, North Holland Publishing Co., N.Y. (1978)). In a typical immunometric assay, a quantity of unlabeled antibody is bound to a solid support that is insoluble in the fluid being tested. After the initial binding of antigen to immobilize antibody, a quantity of detectably labeled second antibody (which may or may not be the same as the first) is added to permit detection and/or quantitation of bound antigen (see, e.g., Radioimmune Assay Method, Kirkham, et al., ed., E&S Livingstone, Edinburgh (1970)). Many variations of these types of assays are known in the art and may be employed for the detection of either peptide or S protein.

Antibodies to peptide may also be used in the purification of peptide or S protein or in the isolation of virus (see, generally, Dean, et al., Affinity Chromatography, A Practical Approach, IRL Press (1986)). Typically, antibody is immobilized on a chromatographic matrix such as Sepharose 4B. The matrix is then packed into a column and the preparation containing peptide, protein or virus is passed through under conditions that promote binding, e.g., under conditions of low salt. The column is then washed and bound material is eluted using a buffer that promotes dissociation from antibody, e.g., buffer having an altered pH or salt concentration. The eluted material may be then transferred to a buffer of choice and either stored or used directly.

IV. Vaccines

The peptides described above may be formulated into vaccines optionally containing salts, buffers or other substances designed to improve the composition. Sterile solutions may be used as the carrier and preservatives may be added to improve the stability of preparations during storage. Adjuvants may be either combined with compositions prior to administration or administered separately to a subject. Adjuvants can take the form of oil-based compositions, e.g., Fruend's complete and incomplete preparations, mineral salts, e.g., silica, kaolin, or carbon, polynucleotides or saponins. Examples of suitable materials for use in vaccines and methods for formulation are provided in Remington's Pharmaceutical Sciences, (pp. 1324-1341, Mack Publishing Co., Easton, Pa. (1980)).

Vaccines may be stored in a lyophilized form and reconstituted in a pharmaceutically acceptable carrier prior to administration. Alternatively, preparations can be stored in solution. The volume of a single dose, i.e., a unit dose, of the vaccine will vary but, in general, should be between about 0.1 ml and 2.0 ml and more typically between 0.2 ml and 1.0 ml.

Any method for administering the vaccines to a patient which does not result in the destruction of peptides is compatible with the present invention. Generally, administration will be by parenteral means such as intramuscular, subcutaneous or intravenous injection. The dosage and scheduling of administration of vaccines can be determined using methods that are routine in the art. By way of example, the vaccines prepared by the methods described herein may contain from about 10 μg/ml to 10 mg/ml or between 50 and 500 μg/ml per dose. However, dosages higher or lower than these ranges are compatible with the invention. The preparations may be administered by either single or multiple injection.

### EXAMPLES

The coronavirus spike (S) protein mediates infection of receptor-expressing host cells, and is a critical target for antiviral neutralizing antibodies. Angiotensin-converting enzyme 2 (ACE2) is a functional receptor for the coronavirus (SARS-CoV) that causes severe acute respiratory syndrome (SARS). The present example demonstrates that a 193-amino-acid fragment of the S protein (residues 318-510) binds ACE2 more efficiently than the full S1 domain (residues 12-672). This region (i.e. residues 318-510) includes seven cysteines, five of which (residues 366, 348, 419, 467 and 474) are essential for expression or ACE2 association. In addition, point mutations at glutamic acid 452 or aspartic acid 454, interfere with or abolish association with ACE2.

Smaller S-protein fragments, expressing residues 327-510 or 318-490, do not detectably bind ACE2. A point mutation at aspartic acid 454 abolished association of the full S1 domain and of the 193-residue fragment with ACE2. The 193-residue fragment blocked S-protein-mediated infection with an IC₅₀ of less than 10 nM, whereas the IC₅₀ of the S1 domain was approximately 50 nM. These data identify an independently folded receptor-binding domain of the SARS-CoV S protein that constitute a target for neutralizing antibodies against the virus.

A. Experimental Procedures

Construction of S1-Ig, Truncation Variants, and Mutants

A plasmid encoding S1-Ig was generated by amplifying a region encoding residues 12 through 672 from an expression vector containing a codon-optimized form of the full-length S-protein gene (Li, et al., Nature 426:450-454 (2003)), and ligating this region into a previously described vector encoding the signal sequence of CD5 and the Fc domain of human IgGI (Farzan, et al. Cell 96:667-676 (1999)). Truncation variants were generated by inverse PCR amplification, using the S1-Ig plasmid as a template. Mutations within S1-Ig, or within a truncation mutant thereof expressing residues 318-510, were generated by site-directed mutagenesis using the QuikChange method (Stratagene). Two independent plasmids were generated for each variant, sequenced within their coding regions, and assayed.

Purification of S1-protein Variants

293T cells were transfected with plasmids encoding S1-Ig or S1-Ig variants. One day post-transfection, cells were washed in PBS and subsequently incubated in 293 SFM II medium (Invitrogen). Medium was harvested after 48 hours and proteins precipitated with Protein A-Sepharose beads at 4° C. for 16 hours. Beads were washed in PBS/0.5M NaCl, eluted with 50 mM sodium citrate/50 mM glycine (pH2), and neutralized with NaOH. Purified proteins were concentrated with Centricon filters (Amicon) and dialyzed in PBS.

Binding and Flow Cytometry

293T cells were transfected with a previously described plasmid encoding ACE2 ((Li, et al., Nature 426:450-454 (2003)) or with vector (pcDNA3.1, Invitrogen) alone. Three days post-transfection, cells were detached in PBS/5 mM EDTA and washed with PBS/0.5% BSA. S1-Ig or variants thereof were added to 10⁶ cells to a final concentration of 250 nM, and the mixture was incubated on ice for one hour. Cells were washed three times with PBS/0.5% BSA, then incubated for 30 minutes on ice with anti-human IgG FITC conjugate (Sigma; 1:50 dilution). Cells were again washed with PBS/0.5% BSA. Binding of IgG-tagged viral proteins to 293T cells transfected with ACE2-expressing plasmid was detected by flow cytometry. The mean value of the binding of S1-Ig or variants with the ACE2-transfected cells was subtracted from that of the mock-transfected cells and normalized to that of S1-Ig.

Immunoprecipitation of Soluble ACE2

293T cells transfected with a previously described plasmid expressing soluble ACE2 (Li, et al., Nature 426:450-454 (2003)) were metabolically labeled with [³⁵S]-cysteine and -methionine. Labeled medium was harvested three days post-transfection. 0.5 ml of soluble-ACE2-containing medium was incubated for 15 minutes on ice with 25 pmol of purified S1-Ig or variants, to a final concentration of 50 nM. 20 μl of Protein A-Sepharose was added to the mixture, which was then incubated for one hour at room temperature. Protein A-Sepharose beads were washed 3 times with PBS/0.1% NP40, and once with PBS. Protein was analyzed by SDS-PAGE and quantified by phosphorimaging using ImageQuant software.

Infection Assay with S-protein-pseudotyped Virus

293T cells were transfected with a plasmid encoding SARS-CoV S protein or VSV-G, together with a previously described plasmid encoding the genome of simian immunodeficiency virus (SIV), modified by deletion of the env gene and by replacement of the nef gene with that for green fluorescent protein (GFP) (Bannert, et al., J. Virol. 74:10984-10993 (2000)). Supernatants of transfected cells were harvested, and viral reverse-transcriptase activity was measured. Supernatants containing S-protein- or VSV-G-pseudotyped SIV were added to ACE2- or mock-transfected 293T cells in the presence or absence of the indicated concentrations of S1-Ig or of the 12-327 or 318-510 variants thereof. Media was changed the following day and GFP expression in infected cells was measured two days later by flow cytometry.

B. Results

A protein in which the S1 domain of the SARS-CoV S protein was fused to the Fc region of human IgG1 has been shown to associate with ACE2-expressing cells and to precipitate ACE2 ((Li, et al., Nature 426:450-454 (2003)). To identify the receptor-binding domain of the S protein, this fusion protein, S1-Ig, was sequentially deleted at the N- and C-termini of the S1 domain to make a total of 12 additional variants. Each variant expressed efficiently and could be readily purified using Protein A-Sepharose beads. S1-Ig and truncation variants thereof were used to precipitate a metabolically labeled and soluble form of ACE2. In contrast to an analogous MHV S-protein truncation variant, which efficiently binds the MHV receptor CEACAM1 (Kubo, et al., J. Virol. 68:5403-5410 (1994)), the S1-Ig variant containing S1-domain residues 12-327 did not associate with ACE2. Neither did another expressing residues 12-481, whereas variants expressing residues 12-510 and 12-572 efficiently bound soluble ACE2. These data indicate that residues 511 to 672 at the C-terminus of the S1 domain do not contribute significantly to ACE2 association.

Removal of residues 12 through 260 from the S1-Ig N-terminus had no effect on ACE2 association. Variants expressing residues 298-510 and 318-510 efficiently bound S protein. The 318-510 variant precipitated ACE2 more efficiently than did the full S1 domain. However, two variants expressing slightly smaller fragments of the S1 domain (residues 318-490 and 327-510) did not detectably precipitate ACE2. These data imply that some residues from 318 to 326 and from 491 to 509 contribute either directly to the association of the S1 domain with ACE2, or to the correct folding of the receptor-binding domain.

The ability of each S1-Ig truncation variant to precipitate soluble ACE2 over several experiments was examined. A good correlation is observed between these two binding assays. We note that, under conditions used here, flow cytometry more sensitively detects low-affinity associations with ACE2, whereas precipitation better reveals differences among efficiently binding variants.

We further examined the ability of the S1-Ig variant containing residues 318-510 to bind ACE2 with higher affinity than does full-length S1-Ig. A 50 nM concentration of S1-Ig was compared with varying concentrations of the 318-510 variant. The same concentration (50 nM) of 318-510 precipitated more than twice as much ACE2 as did S1-Ig. A 25-nM concentration of 318-510 precipitated the same amount of soluble ACE2 as did 50 nM S1-Ig. The data imply that the 318-510 variant binds ACE2 at least twice as efficiently as does S1-Ig.

We also investigated the ability of S1-Ig and the 318-510 variant to block S-protein-mediated infection. To do so, we utilized a system in which a lentivirus expressing green fluorescent protein and pseudotyped with the SARS-CoV S protein infects 293T cells stably expressing ACE2. Incubation of 293T cells with the 12-327 variant had no effect on infection, consistent with the inability of this variant to bind ACE2. In this assay, S1-Ig inhibited infection by S-protein-pseudotyped lentivirus with an IC₅₀ of approximately 50 nM, whereas the 318-510 variant blocked infection by the same virus with an IC₅₀ of less than 10 nM. The 318-510 variant did not substantially interfere with infection of lentivirus pseudotyped with the VSV-G protein, which mediates entry independently of ACE2. Fluorescent microscopic fields of view were examined in the presence of 250 nM of the 12-327 or 318-510 variants. Many fields lacked observable green cells in the presence of the 318-510 variant.

We asked whether the difference in the abilities of the 318-510 and 327-510 variants to bind ACE2 was a consequence of the loss of cysteine 323 in the latter variant. It was found that this is not the case. A series of point mutations was made in which each of the seven cysteines within 318-510 was altered to alanine. The variant in which cysteine 323 was altered bound ACE2 as efficiently as 318-510 itself. Alteration of cysteine 378 also had little effect on binding; however, a combination of mutations at residues 323 and 378 resulted in a construct with decreased ability to bind ACE2. Alteration of cysteine 366 or 419 substantially impaired expression of the 318-510 variant. Similar alterations of cysteines 348, 467, and 474 prevented efficient precipitation of ACE2 without a major effect on expression. These data indicate that determinants between 318 and 326 other than cysteine 323 contribute directly or indirectly to ACE2 association.

Finally, we explored the ability of some acidic residues between 318 and 510 to contribute to ACE2 association, focusing on a region highly divergent among coronavirus S proteins. Glutamic acid 452 and aspartic acids 454, 463, and 480 were individually altered to alanine in the 318-510 variant (E452A, D454A, D463A, and D480A, respectively). These 318-510 variants were assayed for their ability to bind ACE2. No effect was observed with the D480A alteration. The E452A and D454A 318-510 variants precipitated approximately 1% and 10%, respectively, of the ACE2 precipitated by the wild-type 318-510 variant. The full S1 domain, when mutated at E452, precipitated ACE2 with an efficiency similar to that of the 318-510 variant bearing the same mutation. The D454A alteration completely abolished ACE2 association both in the 318-510 variant and in the full-length S1 domain, without affecting expression of either protein. These data suggest that ACE2 interacts with the SARS-CoV S domain in the vicinity of aspartic acid 454.

Discussion

The studies described here localize the SARS CoV S-protein receptor-binding domain. A series of truncation variants of the S1 domain, fused to the Fc region of human IgG1, were assayed for their ability to associate with ACE2 on the surface of transfected cells, and to immunoprecipitate soluble ACE2. The smallest fragment that retained ACE2 association was composed of residues 318-510 and bound ACE2 more efficiently than did the full-length S1 domain, whereas slightly smaller fragments did not. The higher affinity of the 193-residue fragment raises the possibility that other regions of the S protein partially mask this receptor-binding domain. Alternatively, the receptor-binding domain described here may simply be more soluble or better folded than the S1 protein, which includes regions that may contact the S2 domain or other S proteins in the trimeric complex. The 193-amino-acid receptor-binding region also more efficiently blocked S-protein-mediated infection of ACE2-expressing cells than did the full S1 domain, presumably due to its greater affinity for ACE2. Further study of this fragment may therefore provide insight into development of therapeutics that block SARS-CoV infection.

We also investigated the role of cysteines and some acidic residues within the 193-residue fragment. We found that most of the seven cysteines contributed to expression or to ACE2 association, and were unable to immediately identify non-essential or unpaired cysteines within this variant. We did, however, identify two acidic residues, glutamic acid 452 and aspartic acid 454, that appear to make an important contribution to S1-protein interaction with ACE2. Although conformational changes due to alteration of these residues cannot be excluded, the observations that variants containing these mutations expressed as efficiently as those bearing wild-type sequences, and that these mutations had nearly identical effects on the 318-510 variant and the full-length S1 domain, suggest that one or both of these residues contribute directly to ACE2 association.

At this time, public-health measures have successfully controlled transmission of SARS-CoV, but it remains unclear whether SARS will reemerge as a threat to human health. Fortunately, several observations suggest that the development of a vaccine against this virus will be less challenging than, for example, the development of an anti-HIV-1 vaccine. SARS-CoV is transmitted more rapidly than an anti-viral antibody response can develop; this suggests that, in contrast to the HIV-1 envelope glycoprotein, the S protein may do little to cloak its receptor-binding domain. Also, again in contrast to HIV-1, and due either to the fidelity of the RNA polymerase or to the rate of transmission, surprisingly little variation has been observed in S-protein genes obtained from separate patients. Together, these observations suggest that a subunit vaccine that includes the S-protein receptor-binding domain described here should be effective in the control of virus transmission.

All references cited herein are fully incorporated by reference. Having now fully described the invention, it will be understood by those of skill in the art that the invention may be performed within a wide and equivalent range of conditions, parameters and the like, without affecting the spirit or scope of the invention or any embodiment thereof.

1. A substantially pure angiotensin-converting enzyme 2 (ACE2) binding peptide comprising an amino acid sequence matching that of SEQ ID NO:1, wherein:
a) said sequence begins at its N terminus at an amino acid in SEQ ID NO:1 selected from amino acid 318-326; and
b) said sequence extends toward the C terminus to at least amino acid 491 and no further than amino acid 672 in SEQ ID NO:1.

2. The peptide of claim 1, wherein said sequence begins at amino acid 318.

3. The peptide of claim 1, wherein said peptide extends to at least amino acid 510.

4. The peptide of claim 1, wherein said peptide extends no further than amino acid 510.

5. The peptide of claim 2, wherein said peptide extends to at least amino acid 510.

6. The peptide of claim 2, wherein said peptide extends no further than amino acid 510.

7. The peptide of claim 1, wherein said peptides begins at amino acid 318 and ends at amino acid 510.

8. A substantially pure angiotensin-converting enzyme 2 (ACE2) binding peptide comprising an amino acid sequence matching that of SEQ ID NO:1, wherein:
a) said sequence ends at its C terminus at an amino acid in SEQ ID NO:1 selected from amino acid 491-510; and
b) said sequence extends toward the N terminus to at least amino acid 326 and no further than amino acid 12 in SEQ ID NO:1.

9. The peptide of claim 8, wherein said sequence ends at amino acid 510.

10. The peptide of claim 8, wherein said peptide extends to at least amino acid 318.

11. The peptide of claim 8, wherein said peptide extends no further than amino acid 318.

12. The peptide of claim 9, wherein said peptide extends to at least amino acid 318.

13. The peptide of claim 9, wherein said peptide extends no further than amino acid 318.

14. The peptide of either claim 1 or claim 8, wherein said peptide is detectably labeled.

15. The peptide of claim 14, wherein said peptide is labeled either radioactively or with a fluorescent tag.

16. A fusion polypeptide consisting essentially of the peptide of either claim 1 or claim 8 fused to a marker amino acid sequence.

17. The fusion polypeptide of claim 16, wherein said marker amino acid sequence is that of the Fc fragment of IgG.

18. The fusion polypeptide of claim 17, wherein said IgG is human IgG.

19. A substantially pure polynucleotide consisting essentially of nucleotides encoding the peptide of any one of claims 1, 8, or 16-18.

20. A vector comprising a promoter operably linked to the polynucleotide of claim 19.

21. A host cell transformed with the vector of claim 20.

22. A method for inhibiting the binding of the S protein of SARS to a host cell receptor comprising contacting said receptor with the peptide of any one of claims 1, 8 or 18.

23. A method for determining whether a cell has a receptor that binds to the SARS S protein, comprising:
a) incubating said cell with a solution comprising the peptide of any one of claims 14-18;
b) removing said solution from said cells; and
c) assaying the cell of step b) to determine the amount of peptide bound.

24. A method of assaying a test compound for its ability to inhibit the binding of the SARS S protein to its receptor, comprising:
a) incubating a host cell expressing a receptor that binds to said S protein with a solution comprising:
i) the peptide of any one of claims 14-18;
ii) said test compound;
b) after the incubation of step a), removing said solution from said cells;
c) assaying the cells of step b) to determine the amount of label present;
and
d) comparing the results of step c) to results obtained using cells prepared in the same manner but which are incubated in the absence of said test compound.

25. A fluorescence-activated cell sorting (FACS) assay for identifying cells having a receptor that binds the S protein of SARS, comprising:
a) incubating cells with a solution comprising the peptide of claim 1 or claim 8, wherein said peptide is labeled in a manner that permits detection by fluorometry;
b) removing said solution from said cells; and
c) assaying the cells from step b) to determine the amount of label present.

26. The assay of claim 25, wherein said peptide is detectably labeled by being expressed as a fusion polypeptide with a marker amino sequence and wherein said cells are assayed by binding a fluorescently tagged molecule to said marker amino acid sequence prior to fluorometry.

27. The assay of claim 26, wherein said marker amino acid sequence is that of the Fc fragment of IgG and wherein said fluorescently tagged molecule is a labeled antibody.

28. A FACS assay for determining the ability of a test compound to inhibit the binding of the SARS S protein to its receptor, comprising:
a) incubating cells that express a receptor that binds with specificity to said SARS S protein in a solution comprising:
i) said test compound;
ii) the peptide of either claim 1 or claim 8, wherein said peptide is labeled in a manner that permits detection by fluorometry;
b) removing said solution from said cells;
c) assaying the cells from step b) to determine the amount of label present; and
d) comparing the results of step c) to results from cells prepared in the same manner but which are incubated in the absence of said test compound.

29. The assay of claim 28, wherein said peptide is detectably labeled by being expressed as a fusion polypeptide with a marker amino acid sequence and wherein said cells are assayed by binding a fluorescently tagged molecule to said marker amino acid sequence prior to fluorometry.

30. The assay of claim 28, wherein said marker amino acid sequence is that of the Fc fragment of IgG and wherein said fluorescently tagged molecule is a fluorescently labeled antibody.

31. An antibody made by the process of injecting an animal capable of making antibodies with the peptide of either claim 1 or claim 8.


SEQUENCE LISTING (2 sequences)

SEQ ID NO: 1 - protein, 1255 aa, SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - protein, 193 aa, SARS coronavirus
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATV